SEQUENCE LISTING (12 sequences)

SEQ ID NO: 1 - DNA, 29 bp, Artificial Sequence
  synthetic oligonucleotide primer
ataggatccaccatggaggagccgcagtc

SEQ ID NO: 2 - DNA, 26 bp, Artificial Sequence
  synthetic oligonucleotide primer
ataggatccatgtcagtctgagtcag

SEQ ID NO: 3 - DNA, 1187 bp, Human
ccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcatgtttgtgcctgtcctgggagagaccggcgcacagaggaagagaatctccgcaagaaaggggagcctcaccacgagctgcccccagggagcactaagcgagcactgtccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacat

SEQ ID NO: 4 - DNA, 9 bp, Artificial Sequence
  synthetic oligonucleotide sequence (Kozak regular sequence)
ccaccatgg

SEQ ID NO: 5 - DNA, 1187 bp, Artificial Sequence
  synthetic nucleotide sequence encoding human p53 gene
ccaccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcatgtttgtgcctgtcctgggagagaccggcgcacagaggaagagaatctccgcaagaaaggggagcctcaccacgagctgcccccagggagcactaagcgagcactgtccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 6 - DNA, 20 bp, Artificial Sequence
  synthetic oligonucleotide primer
agcactgtccaacaacacca

SEQ ID NO: 7 - DNA, 20 bp, Artificial Sequence
  synthetic oligonucleotide primer
acatccactttgcctttctc

SEQ ID NO: 8 - DNA, 20 bp, Artificial Sequence
  synthetic oligonucleotide primer
gggacagcatcaaatcatcc

SEQ ID NO: 9 - DNA, 20 bp, Artificial Sequence
  synthetic oligonucleotide primer
tcgtttctcagcagctgttg

SEQ ID NO: 10 - DNA, 20 bp, Artificial Sequence
  synthetic oligonucleotide primer
catctgaactcaaagcgtgg

SEQ ID NO: 11 - DNA, 2192 bp, Artificial Sequence
  nucleotide sequence of eukaryotic gene expression vector
gagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcactcattctattggctgagctgcgttctacgtgggtataagaggcgcgaccagcgtcggtaccgtcggaattgtgagcggataacaattggtaccgtcgcagtcttcggtctgacaacgcagatcgaattgggcgtctaaccagtcacagtcgcaaggtaggctgagcaccgtggccaccgtaggggcggcagcgggtggcggtcggggttgtttctggcggaggtgctgctgatgatgtaattaaagtaggcggtcttgagacggcggatggtcgaggtgaggtgtggcaggcttgagatcgatctggccatacacttgagtgacaatgacatccactttgcctttctctccacaggtgtccactcccaggtccaaccgaattcaagctgctagcaaggatccaccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcatgtttgtgcctgtcctgggagagaccggcgcacagaggaagagaatctccgcaagaaaggggagcctcaccacgagctgcccccagggagcactaagcgagcactgtccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacatggatccagcttgtcgacttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgaagatcc

SEQ ID NO: 12 - DNA, 3743 bp, Artificial Sequence
  nucleotide sequence for LacZ gene
aagcttgggatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctggtaccggtgggtgaagaccagaaacagcacctcgaactgagccgcgatattgcccagcgtttcaacgcgctgtatggcgagatcgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggaacttgtttattgcagcttataatggttacaaataaagcaatagcatcacaaatttcacaaataaagcatttttttcactgcattctagttgtggtttgtccaaactcatcaatgtatcttatcatgtctggatcc

(19) United States
(12) Patent Application Publication
(10) Pub. No.: US 2010/0034775 A1
(43) Pub. Date: Feb. 11, 2010

(54) RECOMBINANT ADENOVIRUS COMPRISING RECOMBINANT khp53 GENE AND THE PREPARATION METHOD AND USES THEREOF

(75) Inventor: Qingjun Xie, Guandong Prov. (CN)

Correspondence Address: SCULLY SCOTT MURPHY & PRESSER, PC, 400 GARDEN CITY PLAZA, SUITE 300, GARDEN CITY, NY, 11530, US

(73) Assignee: SHENZHEN WELL. D GENETIC ENGINEERING CO., LTD., Shenzhen, Guangdong (CN)

(21) Appl. No.: 12/520,615

(22) PCT Filed: Dec. 20, 2006
(86) PCT No.: PCT/CN2006/003506

§ 371 Date: Jun. 22, 2009

(51) Int. Cl. A61K 35/76; C12N 15/00; C12N 7/00; C12N 15/87

(52) U.S. Cl. 424/93.2; 435/320.1; 435/235.1; 435/455

(57) ABSTRACT

A recombinant adenovirus vector comprises eukaryotic cell promoter, operator, Kozak sequence, exogenous gene and PolyA signal sequence from 5′ to 3′ in the adenovirus vector backbone. Optionally, introns are inserted between the operator and Kozak sequence, and the exogenous gene is p53 gene. A recombinant adenovirus obtained by co-transfection of the adenovirus vector and adenovirus backbone is also included in the invention. Also disclosed are the preparation methods and uses of the recombinant adenovirus vector and the recombinant adenovirus, respectively. The present invention further discloses a pharmaceutical composition for treating cancers, which comprises the recombinant adenovirus.

### FIELD OF THE INVENTION

The invention belongs to the field of genetic engineering. In particular, the invention relates to a recombinant virus vector for a human tumor suppressor gene, especially to a Kozak sequence-directed recombinant adenovirus vector for a human p53 tumor suppressor gene, and also to a pharmaceutical composition comprising said recombinant adenovirus vector. The invention further discloses the preparation methods and uses of the recombinant adenovirus vector.

### BACKGROUND OF THE ART

### 1. Tumor Gene Therapy

Gene therapy is a molecular medical therapy aiming for radically curing diseases, which comprises the following steps: introducing a gene or DNA (RNA) fragment with therapeutic function into human target cells in a certain way; and modulating the expression of the gene at a molecular level in accordance with various pathological changes resulted in diseases, so as to exert the therapeutic function.

Tumor gene therapy has been rapidly developed for the past more than 10 years. In the United States, there founded 10 large gene therapy companies in succession around 1995. The number of professional gene therapy companies reached about 30 in 1999, and almost 100 in 2000. In 2004, among the 240 or more gene therapy companies in the whole world, 75 of them were researching and developing tumor gene therapies. Until the end of July 2004, the number of approved clinical experimental procedures of gene therapies reached 987 in 23 countries in the world. Most of those procedures (656 of the 987 procedures), accounting for 66.5%, targeted at tumors. The administration routes included intra-tumor tissue, intravenous, intra-hepatic artery, subcutaneous, intramuscular, intra-bladder, and intraperitoneal, etc. The first commercially available gene therapeutic medicine in the world was used to treat tumors.

The research and application of a gene therapy is derived from the treatment of genetic diseases, but the tumor gene therapy has been developed far beyond genetic diseases therapy recently. The major reason is that the incidence of tumors and the number of patients with tumors are much greater, while compared with those of genetic diseases. According to the report by the World Health Organization, the number of patients with newly diagnosed malignant tumors was 10.1 millions in 2000, wherein 0.62 million patients died. 22.4 million patients are suffering from malignant tumors currently. During the past 10 years, the global morbidity of malignant tumors was increased 22%, which indicated that the malignant tumor had become one of the major common diseases severely shortening human lives and lowering life quality. The malignant tumors not only dramatically impact human health, but also bring significant mental and physical harm to patients. A secondary reason is that there exists a large amount of widely ranged therapeutic gene types, which are not only restricted within defected or mutated genes as in genetic diseases. Any gene, such as a tumor suppressor gene, an immune enhancer gene, a suicide gene, a neovascularization suppressor gene, etc can be utilized, as long as it enables the treatment purpose of tumors but only slightly hurts normal somatic cells. The third reason is that the gene therapy of tumors does not require constant expression of ectogenic genes. Periodical expression or even temporary expression of the gene can kill tumor cells as well. The final reason is that most of the gene therapies of tumors involve introducing therapeutic genes into tumor cells directly, which requires less accuracy for modulating the expression of therapeutic genes than genetic diseases does.

2. Investigation on Human p53 gene

Lane and Crawford et al. (1979) (Lane D P, Crawford L V. T antigen is bound to a host protein in SV40 transformation cells [J]. Nature, 1979/278/570 P 261-263) found a p53 gene in the cell transfected by a SV40 large T antigenic gene, and believed it was an oncogene. After that, through studies, Finlay et al. (Levine A J, Momand J, Finlay C A. The p53 tumor suppressor gene [J]. Nature, 1991, 351(3):453) discovered that if there existes a wild type p53 gene in the cell transfected by an oncogene such as myc or ras, the growth of said cell would be inhibited. Therefore, it is suggested that the p53 gene is a cancer suppressor gene.

Human p53 gene is located at 17p13.1 region of the 17^{th} chromosome, with a length of 20303bp in the genomic DNA. Human p53 gene comprises 11 exons and 10 introns, which encodes a protein of 393 amino acid residues, i.e. P53 protein. According to the functions, P53 protein is divided into 5 parts: N-terminal transcription activation region (1-42 aa), signal region (64-92 aa), DNA binding region (102-292 aa), tetramer region (324-355 aa), and C-terminal non-specific DNA binding region (370-393 aa). The N-terminal transcription activation region can bind to a series of protein structures (such as MDM2 TBP TAFs), thereby adjusting the transcription function of p53. The signal region enriches proline, which comprises 5 repeats of proline-X-X-proline, this region is not necessary for the transcription activation, but it is essential for inducing apoptosis. The DNA binding region recognizes sequence-specific DNA sequences. The tetrame region can inhibit the growth of cancer cells. The C-terminal non-specific DNA binding region can bind to non-specific DNA sequences. The basic function of p53 gene is acting as a nucleic transcription factor. The anti-tumor bioactivity thereof mainly includes blocking tumor cell cycles such as G1/S and G2/M; inducing tumor cell apoptosis; suppressing the angiogenesis of tumors; enhancing the immunogenicity of tumor cells; inhibiting the infiltration and metastasis of tumor cells; and raising the sensitivity of tumor cells to radiochemical therapies and thermotherapies, etc.

p53 gene mutation is one of the major reasons for the genesis of many tumors. The gene mutation mainly includes point mutation, allelic deletion, gene rearrangement, and inactivation by binding to tumor virus oncoproteins, etc. It was reported that p53 gene mutation happened in 50% of the approximately over 200 different types of tumors. It has been disclosed that there are 4 mutation hotspots located at exons 5-8 in p53 gene. Although the mutation spectra of p53 gene in tumors generated in different tissue organs are different, around 90% mutations focus in those hotspots. Such hotspots encode amino acid sequences at position 132-143, 174-179, 236-248 and 272-281, respectively.

In many types of human tumors, for example in oesophageal cancer, lung cancer, gastric cancer, liver cancer, colonal and rectal cancer, pancreatic cancer, breast cancer, ovarian cancer, thyroid cancer, osteosarcoma, rhadomyosarcoma, neuroblastoma, about 50% p53 genes mutate. In melanoma (primary), the mutation rate of p53 gene is up to 97%. p53 gene mutation occurs in all types of lung cancer. For instance, p53 gene mutation occurs in about 52% of non-small cell lung carcinoma and 90% of small cell lung carcinoma. p53 gene also mutates in some leukaemia, cerebroma, multiple myeloma, astrocytoma. Studies showed that p53 mutation rate was higher in refractory tumors and lower in relatively tractable tumors. Moreover, p53 gene mutation closely relates to the progress of diseases, metastasis of tumors and the sensitivity and prognosis of radiochemical therapies.

### 3. Investigation on Adenovirus Vector

The structure of adenovirus is the characteristic icosahedron capsid, with no envelope, a diameter of 70˜90 nm, and 252 capsomers, in which the icosahedron parietal angled capsomers are 12 pentons with 2 fibers each. Besides the pentons, there also exists 240 non-parietal angled capsomers, called hexons. The genome of adenovirus is a linear double-stranded DNA of ˜36 kb binding with a terminal protein (TP) covalently at its 5′-end. There are also inverted terminal repeats (ITRs) located at the 5′-end. Virus DNA closely binds to core protein VII and a small peptide called mu. Another protein V coats the resultant DNA-protein complex, and protein VI provides a structural linkage between the DNA-protein complex and the capsid. To prepare an adenoviral genome that acts as a gene therapeutical vector, its E1 region and/or E3 region are usually deleted, and an exogenous gene expression cassette is added into the position of the E1 region. The resulted recombinant adenovirus is a replication-defective virus, which means it can not be replicated in a normal cell, but can only be replicated and generated in a specific cell (such as 293 cell).

The characteristics of an adenovirus vector include: larger capacity of vectors (>7.5 kb), extremely high transfection rate (the in vitro transfection rate of human tumor cells reaches 100%), high productive efficiency (viruses can be amplified hundreds of times by producing cells), non-integrative infection of cells, genetic non-toxicity, strong infecting ability (both cleavage and non-cleavage cells may be infected), high expression of an exogenous gene. Those characteristics enable adenoviruses to be especially suitable for gene therapy of tumors and industrial production of the products thereof.

The evolvement of the method for preparing the recombinant adenovirus vector strain includes: two-plasmid co-transfection method, Ad-Easy method, and AdMax method. The two-plasmid co-transfection method is a less efficient method used in an earlier stage, which comprises the following steps: co-tranfecting packaging cells, 293 cells with a shuttle vector small plasmids carrying a target gene and a large plasmid carrying the adenoviral genome; generating the genome of recombinant adenovirus vector by random homologous recombination of both said plasmids in the 293 cells; and packaging into the virions in the 293 cells. Ad-Easy method is a more efficient method, comprising the following steps: transforming bacteria carrying most parts of the adenoviral genome with a adenovirus shuttle plasmid carrying a cloned exogenous gene; generating a plasmid containing the recombinant adenovirus vector genome by recombination under the action of bacterial recombination enzyme Cre; amplifying and extracting the plasmid after screened for resistance; transfecting 293 cells with the plasmid after digested and purified; and packaging into the recombinant virons in the 293 cells. AdMax method is the method with the highest packaging efficiency so far, which comprises the following steps: co-transfecting 293 cells with an adenovirus shuttle plasmid carrying a cloned exogenous gene and a packaging plasmid carrying the most parts of adenoviral genome; and generating the recombinant adenovirus by recombination under the action of recombination enzymes (Ng P, Parks R J, Cummings D T, et al. An enhanced system for construction of adenoviral vectors by the two-plasmid rescue method [J]. Hum Gene Ther. 2000,11 (5): 693-699.).

The expression of an adenovirus vector carrying an exogenous gene often results in a certain level of cytotoxicity. Sometimes, even a medium expression level of a gene without cytotoxicity may clearly block the replication and amplification of an adenovirus vector. As a result, it is very difficult to create a recombinant adenoviron with transfective activity, or to amplify it to obtain a high titer value. In such circumstances, the virus highly expressing a target protein can not be obtained by the virus monoclonal screening method either. For example, the adenovirus expressing a glycoprotein is usually difficult to produce virion, and so are some adenovirus vectors expressing a certain DNA binding protein or enzyme. Therefore, it is hard to estimate the titer value and the yield of a certain adenovirus strain in advance.

The preparation of the adenovirus vectors described above can be achieved by AdMax™ Hi-IQ adenovirus vector system. Based on AdMax, AdMax™ Hi-IQ adenovirus vector system utilizes a lac operator to control the transcription of target genes, and packages adenovirus vectors on the 293 cells expressing lac repressors, thereby inhibiting the expression of the target genes on those cells. However, the expression of target genes on other cells is not inhibited.

“Hi-IQ” system can be used for the high yield preparation of an adenovirus vector carrying a target gene providing or potentially providing cytotoxicity. Unlike AdMax system, “Hi-IQ” system includes a 293 cell line expressing a lac repressor (293-IQ cell) and a shuttle plasmid containing a MCMV promoter, an intron and a lac operator, and a helper large plasmid containing the adenoviral genome. The resultant recombinant virus is an E1/E3 deleted replication-defective adenovirus.

The recombinant human p53 adenovirus has been applied to clinically treat malignant tumors for ten years. Although some exciting therapeutic effects have been achieved, in general, the therapeutic effects are still uncertain. The major reason for that is a relative lack of P53 protein expression in tumor cells, which is due to the common utilization of a weaker promoter such as RSV, SV40 or CMV in the eukaryotic expression system of p53 tumor suppressor gene. Some studies indicated that when gene mutation occurred in a normal human cell under various in vivo or in vitro conditions and finally caused the formation of a tumor cell, more than 10000 alternation at a gene level (such as deletion, point mutation and methylization of cancer suppressor gene, and rearrangement, amplification and mutation of pro-oncogene, etc) had been accumulated. However, the action mechanism of tumor suppressor gene p53 is to repress the growth of tumors by blocking cell cycles, facilitating apoptosis of tumor cells, inducing differentiation and aging of cells, and inhibiting angiogenesis in tumors, etc. Therefore, the most significant action feature of p53 is multiple target points, i.e. not only one single action point. For instance, P53 protein, as a transcription factor, directly reacts with a specific DNA sequence, thus regulating the expression of various genes downstream. A genechip research showed that the transcription of 107 genes was activated by P53 protein and thereby the expression thereof was up-regulated, whereas the expression of 54 genes was down-regulated due to the inhibition for their transcription. Also, P53 protein per se can interact with several intracellular proteins. For example, the gene expression product of oncogene mdm2 can specifically bind to P53 protein, thereby inhibiting the function of P53. Accordingly, unlike the plenty of gene changes in tumor cells, after the recombinant human p53 adenovirus gene medicine formulation, used for gene replacement therapy, is utilized to infect tumor cells and is expressed, the expressed amount of P53 protein is little, thus it is difficult to effectively control tumor growth.

However, after we constructed the human p53 recombinant adenovirus shuttle vector with high expression ability, it was found that such a vector was not able to package into a recombinant adenovirus in 293 cells, and thereby to transduct a human p53 gene into a tumor cell, thereby it could not be used to achieve the purpose of a gene therapy. The major reason for that is the equally effective expression of the expression system highly expressing human p53 gene in the packaging cells depletes a large amount of resources used for the protein synthesis in packaging cells (because the packaging process of a recombinant adenovirus is also a process in which a large amount of proteins are synthesized, there forms a competitive relation between them). Moreover, many P53 proteins result in blocking cell cycles of 293 cells, or even apoptosis. Hence, besides the effective expression of genes, the effective packaging of a recombinant adenovirus is also needed.

### DISCLOSURE OF INVENTION

The object of the invention is to provide a recombinant adenovirus, wherein multiple regulation elements improving gene expression, such as MCMV (Murine Cytomegalovirus) promoters, introns, and Kozak sequences, etc, are used comprehensively in an eukaryotic expression system inserted in said recombinant adenovirus, leading human p53 tumor suppressor gene to enter a tumor cell through a recombinant adenovirus vector at different levels and stages of gene expression, directly followed by the mass expression of p53 in the tumor cell, inhibition for the growth of the tumor cell, and apoptosis in the end. Also, an operator, such as E. coli Lac Operator, is inserted into the downstream of MCMV in the eukaryotic expression system, and a lac repressor gene specifically binding to the DNA sequence of E. coli Lac Operator is transferred into 293 packaging cells and expressed stably. In this way, in the packaging process of the recombinant adenovirus, the lac repressor protein expressed in 293 cells is located between MCMV promoter and human p53 tumor suppressor gene, which disables the normal transcription of p53 gene, and clearly represses the expression of an exogenous gene in 293 cells, but does not affect the packaging process of the recombinant adenovirus. The above two methods permit human p53 tumor suppressor gene to be effectively expressed in tumor cells and to be effectively packaged in the transgenetically engineered 293 cells (293IQ cells).

Accordingly, one aspect of the invention relates to a recombinant adenovirus vector, wherein a Kozak sequence and an exogenous gene are inserted between the operator of the adenovirus shuttle vector pDC315(io) or pDC316(io) and a polyA signal sequence.

Another aspect of the invention relates to a recombinant adenovirus vector, wherein a Kozak sequence and an exogenous gene are inserted between the operator of the adenovirus shuttle vector pDC515(io) or pDC516(io) and a polyA signal sequence.

Preferably, said Kozak sequence is CCACCATGG; said exogenous gene is human p53 gene,the sequence thereof is shown as SEQ ID NO:3.

A further aspect of the invention relates to an eukaryotic gene expression system, comprising the sequence of SEQ ID NO:11. Preferably, the sequence thereof is shown as SEQ ID NO:11.

A further aspect of the invention also relates to a recombinant adenovirus, which is obtained by co-transfecting 293IQ cells with said recombinant adenovirus shuttle vector and a adenovirus backbone, wherein when said adenovirus shuttle vector is pDC315(io) or pDC316(io), said adenovirus backbone is pBHGlox(delta)E1,3Cre; when said adenovirus shuttle vector is pDC515(io) or pDC516(io), said adenovirus backbone is pBHGfrt(del)E1,3FLP.

Preferably, said recombinant adenovirus is an adenovirus that was deposited in China Center for Type Culture Collection on Jun. 20, 2005, with a deposit number of CCTCC V200508.

Another aspect of the invention relates to a medicament used for treating cancers, said medicament comprises the recombinant adenovirus of the present invention and optionally, a pharmaceutically acceptable excipient.

A further aspect of the invention also relates to use of said recombinant adenovirus for the manufacture of a medicament treating cancers, wherein said cancer is selected from lung cancer, liver cancer, breast cancer, nasopharyngeal cancer, ovarian caner or cervical cancer.

The present invention also relates to a method for preparing said recombinant adenovirus vector, comprising the step of inserting an exogenous gene linked to a Kozak sequence between the operator of adenovirus shuttle vector pDC315(io), pDC316(io), pDC515(io) or pDC516(io) and a polyA signal sequence in a forward direction.

In addition, the invention also relates to a method for obtaining said recombinant adenovirus, comprising the step of co-transfecting 293IQ cells with a recombinant adenovirus vector and an adenovirus backbone, wherein when said adenovirus shuttle vector is pDC315(io) or pDC316(io), said adenovirus backbone is pBHGlox(delta)E1,3Cre; when said adenovirus shuttle vector is pDC515(io) or pDC516(io), said adenovirus backbone is pBHGfrt(del)E1,3FLP.

In another aspect, there is also provided a method for treating cancers, comprising the step of administering a therapeutic effective dose of the recombinant adenovirus of the invention to a patient. The therapeutic effective dose is conveniently determined by one skilled in the art or a physician in accordance with particular circumstances. Particularly, the invention relates to the following steps: amplifying the coding region of human p53 tumor suppressor gene by PCR with 5′-ATA GGATCC ACCATGG AGG AGC CGC AGT C 3′ (SEQ ID NO:1) as a upstream primer and 5′-ATA GGATCC ATG TCA GTC TGA GTC AG 3′ (SEQ ID NO:2) as a downstream primer; cloning the PCR product into a T vector followed by sequencing in both directions; sub-cloning it into a adenovirus shuttle plasmid (pDC315(io), pDC316(io), pDC515(io) or pDC516(io)) using restriction endonulease BamH I; screening a clone inserted in a forward direction by PCR; co-transfecting 293IQ cells with the clone and the DNA of a adenoviral genomic backbone plasmid (pBHGlox(delta)E1,3Cre or pBHGfrt(del)E1,3FLP); packaging a recombinant adenovirus, i.e. rAdMH-khp53 by site-specific homologous recombination in eukaryotic cells; cloning a positive recombinant adenovirus strain; infecting 293IQ cells by the cloned positive recombinant adenovirus strain; amplifying and purifying; and manufacturing medicines for a gene therapy.

The features of the recombinant adenovirus include:
1. A unique eukaryotic gene expression system structure

First of all, the currently most effective promoter for promoting eukaryotic gene expression in the world, i.e. Murine Cytomegalovirus promoter MCMV, is used. Therefore, gene expression is improved at transcription level. An intron is inserted between the promoter and an exogenous gene, which further increases the stability of mRNA after gene transcription. Secondly, there is a delicately designed Kozak sequence located before the coding region of human p53 tumor suppressor gene, thus further raising the expression amount at protein translation level. Lastly, E. coli lac operator is inserted into the downstream of MCMV The co-function of E. coli lac operator and a 293IQ cell effectively facilitates the eukaryotic gene high expression system to be recombined into an adenovirus vector and to be packaged.
2. The advantages of an adenovirus vector include: high transfecting rate, excellent maneuverability, ability to carry larger genes, capability of preparing an effective viron with higher titer, a wide range of hosts, great safety, and low pathogenicity.
3. The multiple targeting property of an anticancer function of human p53 tumor suppressor gene not only blocks tumor cell cycles, represses the growth of tumor cells, and induces apoptosis of tumor cells, but also induces differentiation and aging of cells, suppresses angiogenesis of tumors, enhances the immunogenicity of tumor cells, inhibits the infiltration and metastasis of tumor cells; and improves the sensitivity of tumor cells to radiochemical therapies and thermotherapies, etc.
4. Human p53 tumor suppressor gene is introduced into a human tumor target tissue cell by adenovirus vector and expressed directly in that cell, which overcomes the difficulty that P53 protein is unable to be prepared into a recombinant genetic engineering product in vitro by a genetic engineering method, because of its instability. Moreover, the introduction and expression of human p53 tumor suppressor gene by adenovirus vector makes no difference with an endogenous P53 protein in modification procedures of protein molecules, such as protein phosphorylation, folding and polymerizing, etc, and brings about the exactly same bioactivity, so as to directly exert the anticancer function for topical tumors.

It is contributed by the invention that the constructed recombinant adenoviruses with the unique structures and features, for example rAdMH-khp53, realize the effective expression of human p53 tumor suppressor gene in a human tumor target tissue cell and the effective packaging of human p53 tumor suppressor gene in a specific 293IQ cell. Therefore, the anticancer function thereof is clearly better than that of some low expression systems, which enables the tumor cells insensitive to a low p53 expression gene therapy to be equally effectively inhibited. Therefore, the treatment becomes more accurate and useful. The inventive human p53 tumor suppressor gene linked to an adenovirus can be expressed directly in an eukaryotic cell, meaning that it can be introduced directly into a solid tumor site and expressed in such a site. That makes the subjects receiving the treatment to become a “factory” for producing P53 tumor suppressor proteins. Such a method is able to overcome some disadvantages, such as instability of proteins, short half-life, low activity, and high cost, etc. Therefore, said treatment becomes a therapy acceptable and affordable by most patients.

The 293IQ cell used in the experiments is obtained by introducing an E. coli lac suppressor gene into a 293 cell. Detailed information refers to Matthews D A, Cummings D, Evelegh C, et al. Development and use of a 293 cell line expressing lac repressor for the rescue of recombinant adenoviruses expressing high levels of rabies virus glycoprotein. Journal of General Virology (1999), 80, 345-353.

The entire content of all references cited herein are incorporated by reference.

### BRIEF DESCRIPTION OF THE DRAWING

FIG. 1 is a technique flow chart for investigation on the recombinant khp53 tumor suppressor gene adenovirus.

FIG. 2 shows the result of an agarose gel electrophoretic analysis of the human p53 gene-coding fragment carrying a Kozak sequence, wherein M: DL2000 DNA Marker; 1: a target fragment of 1206bp; 2: a negative control.

FIG. 3 indicates the result of digestion and electrophoretic identification of a recombinant plasmid, pDC515(io)-khp53, wherein lane M:Lamda/EcoT14I DNA Marker; lane 1: the recombinant plasmid, i.e. pDC515(io)khp53; lane 2: pDC515(io)-khp53/BamHI; lane 3: pDC515(io)-khp53/EcoRI.

FIG. 4 shows the formation of the recombinant adenovirus rAdMH-khp53 strain, wherein A: a negative control; B: the 293IQ cells with CPE; C: the 293IQ cells with complete CPE.

FIG. 5 shows the result of PCR and electrophoretic identification of the recombinant adenovirus rAdMH-khp53, wherein lane M:DL2000 DNA Marker; lane 1: a negative control; lane 8: a positive control; lanes 2-7: the clones under identification.

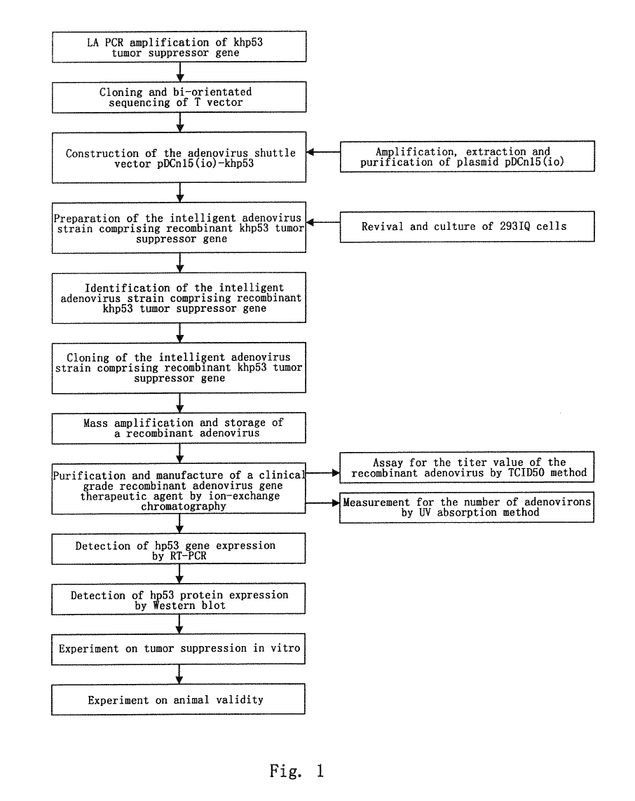

FIG. 6 is a schematic drawing of the genomic structure of the recombinant adenovirus rAdMH-khp53, wherein \textcircled{1} is an intron; \textcircled{2} is a MCMV promoter; \textcircled{3} is an operator; \textcircled{4} is the hp53 coding sequence; \textcircled{5} is a Kozak sequence; \textcircled{6} is SV40PolyA. FIG. 7 is the result of RT-PCR and electrophoretic analysis, showing the expression of p53 mRNA after infecting Saos-2 cells with rAdMH-khp53, wherein
lane 1: the PCR amplification result (a positive control), wherein the template is the RT products of total RNA of A549 cells, the primers are the specific primers in p53 gene coding region (the same primers are used in lanes 2-4);
lane 2: the PCR amplification result (a negative control), wherein the template is the RT products of total RNA of Saos-2 cells;
lane 3: the PCR amplification result, wherein the template is the RT products of total RNA of Saos-2 cells infected with rAdMH-khp53;
lane 4: the PCR amplification result without any template (a blank control);
lane M: DL2000 DNA Marker;
lane 5: the PCR amplification result without any template (a blank control), wherein the primers are GAPDH gene specific primers (the same primers are used in lanes 6-8);
lane 6: the PCR amplification result, wherein the template is the RT products of total RNA of Saos-2 cells infected with rAdMH-khp53;
lane 7: the PCR amplification result, wherein the template is the RT products of total RNA of Saos-2 cells;
lane 8: the PCR amplification result, wherein the template is the RT products of total RNA of A549 cells.

FIG. 8 shows the Western blot result of Saos-2 cells which have been infected with rAdMH-khp53 for 10 hours, wherein lane 1: total proteins of Saos-2 cells; lane 2: total proteins of the Saos-2 cells infected with rAdMH-khp53; lane 3: total proteins of A549 cells.

FIG. 9 shows the anticancer activity of the recombinant adenovirus rAdMH-khp53 in vitro.

FIG. 10 shows the anticancer activity of the recombinant adenovirus rAdMH-khp53 in vivo.

FIG. 11 is a drawing showing the adenovirus shuttle vector, wherein A: pDC315(io), B: pDC316(io), C: pDC515(io), D: pDC516(io).

FIG. 12 is a drawing showing the adenovirus backbone DNA, wherein A: pBHGlox(delta)E1,3Cre; B: pBHGfrt(del)E1,3FLP.

### DETAILED EMBODIMENTS OF THE INVENTION

The present invention is further explained and illustrated by the following examples which are not to be construed to limit the scope of the invention.

The adenovirus vectors pDC315(io) and pDC316(io) used herein and the corresponding adenovirus backbone pBHGlox E1,3Cre were contained in a AdMax HI-IQ Kit H, commercially purchased from Pen Yuan Zheng Yang gene tech Ltd., with a catalog number of PD-01-68. The AdMax HI-IQ Kit H further comprises pFG140 and 293IQ cells. The adenovirus vectors pDC515(io) and pDC516(io) used herein and the corresponding adenovirus backbone pBHGfrt E1,3FLP were contained in a AdMax™HI-IQ Kit J, commercially purchased from Pen Yuan Zheng Yang gene tech Ltd., with a catalog number of PD-01-69. The AdMax™HI-IQ Kit J further comprises pFG140 and 293IQ cells.

### Example 1

### Construction of the Adenovirus Recombinant Comprising Recombinant khp53 Tumor Suppressor Gene

1) Obtaining Human p53 Tumor Suppressor Gene Coding Sequence Carrying a Kozak Sequence (Shorten as khp53):

Two primers were designed according to the published human wild-type p53 cDNA full sequence (SEQ ID No:3): a upstream primer 5′-ATA GGATCC A CCATGG AGG AGC CGC AGT C 3′ (SEQ ID NO:1) and a downstream primer 5′-ATA GGATCC ATG TCA GTC TGA GTC AG 3′ (SEQ ID NO:2). The BamHI digestion site GGATCC and the digestion protective bases ATA were inserted into both primers. The base A was inserted behind the BamHI digestion site GGATCC of the upstream primer, which is followed by the sequence CCATGG AGG AGC CGC AGT C. In this way, the Kozak sequence CCACCATGG (SEQ ID NO:4) is constructed using the bases CC in the BamHI digestion site GGATCC, the base A and an implicit sequence CCATGG of human p53 cDNA. Human p53 tumor suppressor gene coding region was amplified by a PCR reaction, wherein the template was the wild-type human p53 cDNA described above; the enzyme was TaKaRa LA Taq™ DNA polymerase (Bao biotech Ltd. (Dalian)); the reaction condition included: i) denaturalization at 94° C. for 2 min, ii) denaturalization at 94° C. for 30 sec, iii) annealing at 45° C. for 40 sec, iv) extension at 72° C. for 60 sec, v) repeating ii)-iv) for 30 cycles, vi) extension at 72° C. for 5 min, vii) storing at 4° C. A large amount of human p53 gene coding fragments (SEQ ID NO:5) of 1206 bp carrying a Kozak sequence, were obtained after the above reaction. The result of agarose gel electrophoretic analysis was shown in FIG. 2.

2) Cloning and Sequencing of khp53 Tumor Suppressor Gene Coding Sequence:

The PCR product (SEQ ID NO:5) of human p53 gene carrying a Kozak sequence was ligated to a T vector (Shenggong Biological Engineering and Technology Service Ltd of Shanghai) directly. The resultant was used to transform JM109 host bacteria (Fundamental medical research institute, Military medical academy of sciences of Beijing). The transformed bacteria was spread on a LB agar plate containing X-gal, IPTG and Amp. There appeared many blue and white colonies on the plate. The two colour of colonies were picked, 6 clones each, and amplified in a volume of 2 ml, respectively. PCR template was prepared by thermal cleavge the resulted bacterial solution. Using that PCR template, a PCR reaction (The reaction conditions included: i) denaturalization at 94° C. for 2 min, ii) denaturalization at 94° C. for 30 sec, iii) annealing at 50° C. for 30 sec, iv) extension at 72° C. for 30 sec, v) repeating ii)-iv) for 30 cycles, vi) extension at 72° C. for 5 min, vii) storing at 4° C.) was performed to amplilfy the 277 bp specific fragment in human p53 tumor suppressor gene coding region, so as to determine whether human p53 tumor suppressor gene was inserted into those clones. In the PCR reaction, the upstream primer 5′-AGCACTGTCC AACAACACCA 3′ (SEQ ID NO:6)and downstream primer 5′-ATA GGATCC ATG TCA GTC TGA GTC AG 3′ (SEQ ID NO:2)were used. The result showed that the PCR product of the 277 bp fragment didn't exist in the blue clones, meaning the blue clones were blank vectors; whereas the PCR product of the 277 bp fragment did exist in the white clones. Then a small amount of plasmids identified as positive clones by PCR were extracted, digested by BamHI, and electrophoresed. A band of about 1200 bp appeared in front of the band of the linear T vector, confirming there was a human p53 tumor suppressor gene coding fragment both ended with BamHI digestion sites inserted into the T vector. That type of plasmid was called pUCm-khp53, and sequenced in both directions. The sequence of human p53 tumor suppressor gene coding fragment is same as the expected result.

3) Construction of the Recombinant Adenovirus Shuttle Vector pDC515(io)-khp53:

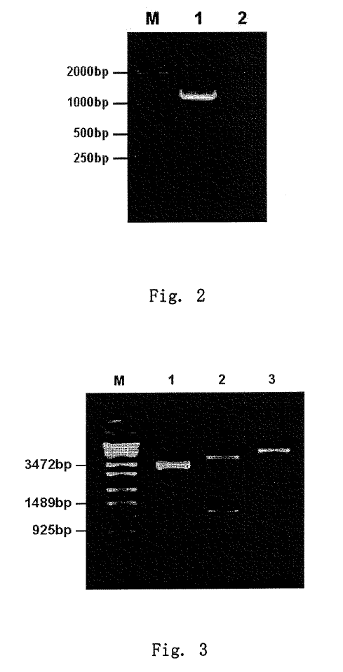

The plasmid pUCm-khp53 and adenovirus shuttle vector pDC515(io) were amplified, extracted and digested with BamHI. The desired gene fragments and the linear vectors were separated by agarose gel electrophoresis. After recovered by gel cutting, the purified target fragments were quantified by UV. The inserted fragments and the vectors were mixed with a ratio of 3:1, and ligated at 16° C. with T4 DNA ligase overnight. The resultant was used to transform competent E. coli. The positive clones were pre-screened out by the method mentioned in above part 2). The inserted direction of a target gene was determined by a PCR reaction, wherein an upstream primer 5′-ACA TCC ACT TTG CCT TTC TC 3′ (SEQ ID NO:7) and a downstream primer 5′-GG GAC AGC ATC AAA TCA TCC 3′ (SEQ ID NO:8) were used, and the reactive conditions included: i) denaturalization at 94° C. for 2 min, ii) denaturalization at 94° C. for 30 sec, iii) annealing at 50° C. for 30 sec, iv) extension at 72° C. for 30 sec, v) repeating ii)-iv) for 30 cycles, vi) extension at 72° C. for 5 min, vii) storing at 4° C. Then a small amount of plasmids identified as positive clones by PCR (i.e. the clones with the fragment inserted in a forward direction) were extracted, digested by BamHI, and electrophoresed. A band of ˜1200 bp appeared in front of the band of the linear shuttle vector, as shown in FIG. 3, confirming there was a human p53 tumor suppressor gene coding fragment both ended with BamHI digestion sites inserted into the vector. Lastly, the positive clones with forwardly inserted genes, named as pDC515(io)-khp53, were stored at −20° C.

4) Preparation of the Adenovirus Strain Comprising Recombinant khp53 Tumor Suppressor Gene:

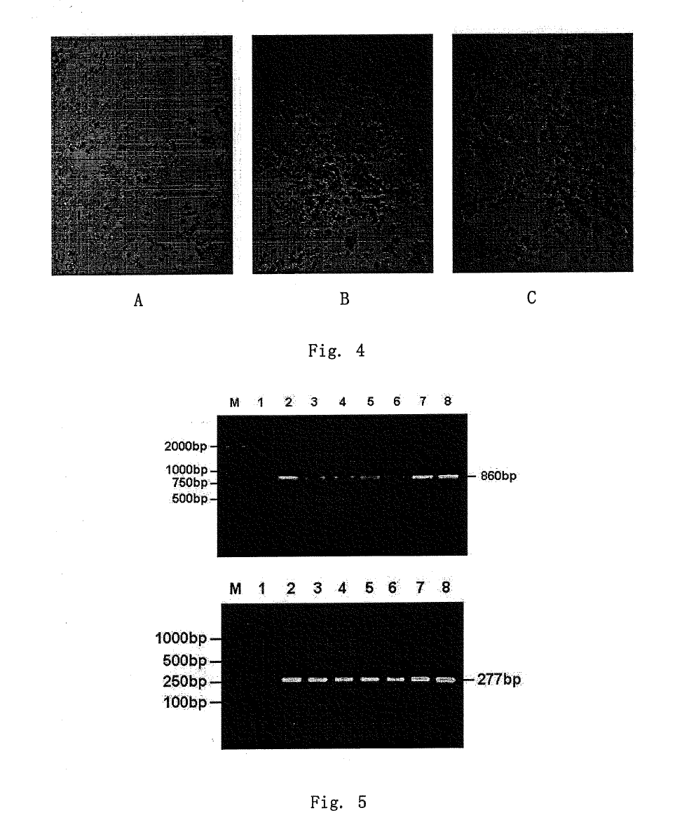

The plasmid of pDC515(io)-khp53, the adenoviral genomic backbone DNA i.e. pBHGfrt(del)E1,3FLP, etc were mass amplified, extracted, purified and quantified (The methods refer to Laboratory Manual of Molecular Cloning, J. Sambrook et al., 2002). After the revival, amplification and plating of 293IQ cells, the 293IQ cells were co-transfected with pDC515(io)-khp53 and the adenoviral genomic backbone DNA (total amount of DNA was 800 ng; and the molar ratio was 1:1) under the induction by Lipofectamine2000 transfection reagent (Zhanchen Biotech Ltd of Guangzhou). And a negative control was set up as well. After 8 days, a cellular pathological effect (CPE) happened to 293IQ cells in the co-transfected wells, whereas the 293IQ cells in the negative control wells grew normally. Therefore, it was primarily concluded that the recombinant adenovirus was generated. In the day 10, a complete pathological change happened to 293IQ cells in the co-transfected wells, as shown in FIG. 4. The pathologically changed cells and the supernatant were collected, repeatedly frozen and melted 3 times at −20° C.˜37° C., and stored at −20° C. for later use.

### Example 2

### Identification, Cloning, Mass Amplification and Purification of the Adenovirus Comprising Recombinant khp53 Tumor Suppressor Gene for Manufacturing a Clinical Grade Therapeutic Agent

1) Identification of the Adenovirus Comprising Recombinant khp53 Tumor Suppressor Gene:

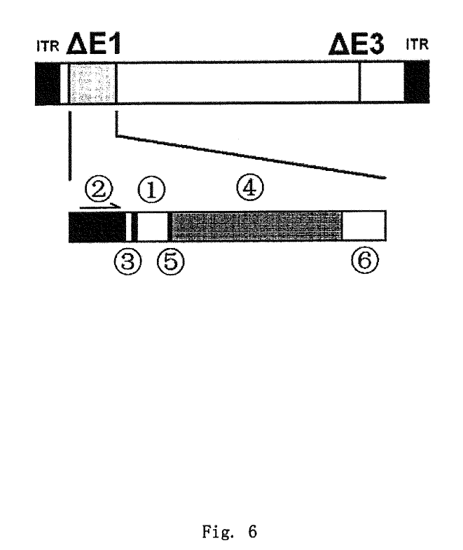

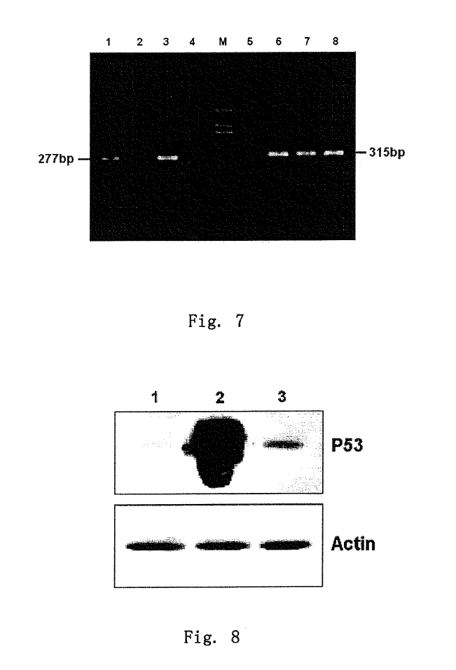

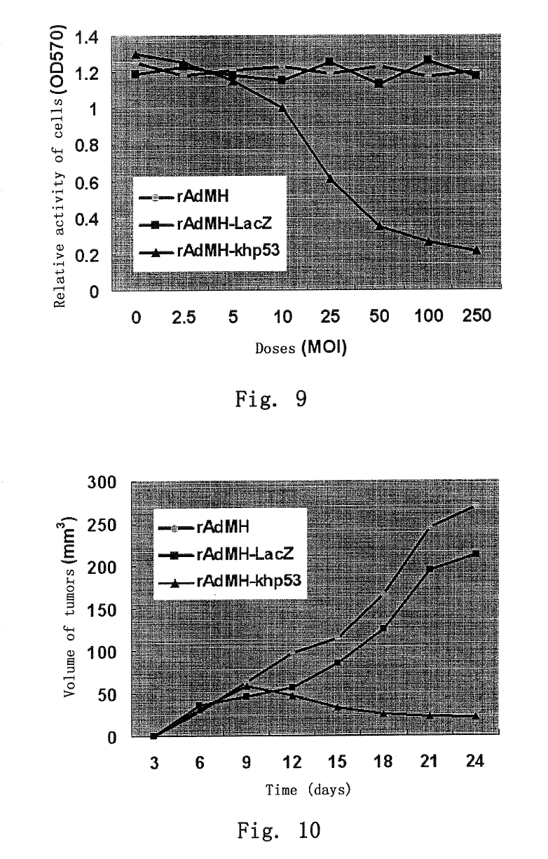

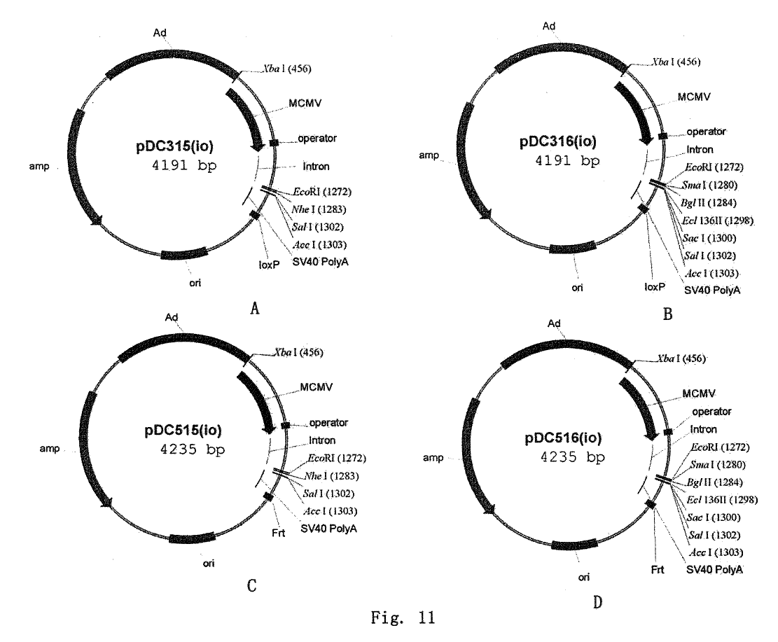

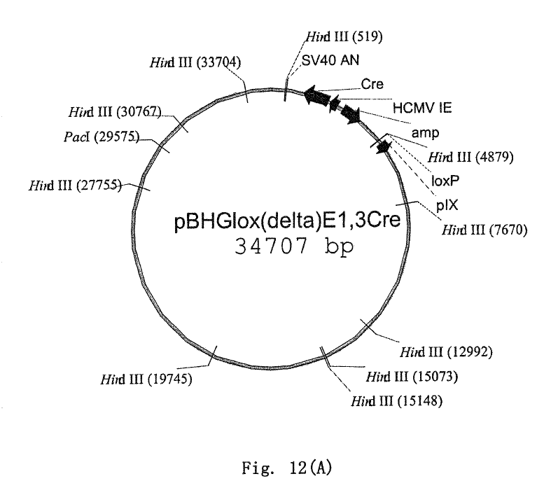

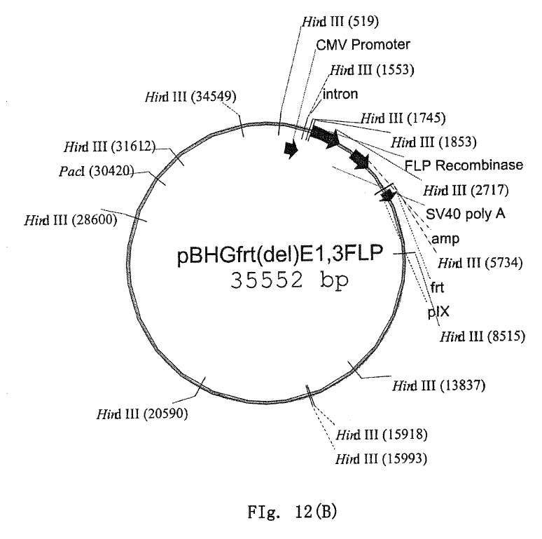

A PCR reaction was carried out to amplify the template prepared by thermal cleavage of recombinant adenovirus, wherein an upstream primer 5′-TCG TTT CTC AGC AGC TGT TG 3′ (SEQ ID NO:9) and a downstream primer 5′-CAT CTG AAC TCA AAG CGT GG 3′ (SEQ ID NO:10) were used; the reactive conditions included: i) denaturalization at 94° C. for 2 min, ii) denaturalization at 94° C. for 30 sec, iii) annealing at 55° C. for 30 sec, iv) extension at 72° C. for 40 sec, v) repeating ii)-iv) for 30 cycles, vi) extension at 72° C. for 5 min, vii) storing at 4° C. If an Ad5 genomic specific fragment (11-13.4 mu, 860 bp) was amplified, the resultant virus described above was identified as a type 5 adenovirus. The 277 bp specific fragment in human p53 tumor suppressor gene coding region was amplified by PCR to determine whether there inserted a human p53 tumor suppressor gene in said type 5 adenovirus, wherein an upstream primer 5′-AGCACTGTCC AACAACACCA 3′ (SEQ ID NO:6) and a downstream primer 5′-ATA GGATCC ATG TCA GTC TGA GTC AG 3′ (SEQ ID NO:2) were used. The results were shown in FIG. 5, wherein all of the 6 clones were type 5 adenoviruses and all carried human p53 tumor suppressor genes.

2) Cloning of the Adenovirus Comprising Recombinant khp53 Tumor Suppressor Gene:

A suspension of 293IQ cells with a concentration of 1×10⁵ cells/ml was prepared with MEM medium. A 96-well plate was inoculated with the suspension, 100 μl/well. The diluted solution of the above recombinant adenovirus was prepared by being diluted in a 10 time series, and utilized to transfect 293IQ cells, respectively. After cultured at 37° C. in a CO₂ incubator for 10 days, the cells were observed under an inverted microscope and the CPE status involving the most diluted diluent was evaluated and recorded. The mono-porous cells generating only one plaque and the supernatant were collected, repeatedly frozen and melted 3 times at −20° C.˜37° C., further amplified on 293IQ cells, and used as a seed of the cloned recombinant adenovirus, with the structure shown in FIG. 6. Such a recombinant adenovirus was named as “E1 and E3 defective type 5 recombinant adenovirus rAdMH-khp53”, shorten as rAdMH-khp53. rAdMH-khp53 was deposited in China Center for Type Culture Collection, Wuhan, China in Jun. 20, 2005, with a deposit number of CCTCC V200508.

### 3) Mass Amplification and Storage of a Recombinant Adenovirus:

Under the condition of 37° C. and 5% CO₂, 293IQ cells were cultured in 100 cell culture plates (medium: MEM+10% FBS) with Φ15 cm. Twenty microliter of the above seed virus solution was not added until the cells reached 80-90% confluence. The cells were returned back to and further cultured, observed under the condition of 37° C. and 5% CO₂. After pathological changes happened to all cells, the suspended cells were pipetted up and down, and the cell suspension was collected in a centrifuge tube. Cells were collected after centrifuged at 800-1000 rpm for 5 min. The cell pellet collected from each 300 cm² suspension were resuspended in 2 ml adenoviral storage reagent (1×PBS²⁺+10% glycerol), repeatedly frozen and melted 3 times at −20° C.˜37° C., and centrifuged at 4° C., 2000 rpm for 10 min. The supernatant was collected, filtered through a 0.45/0.2 μm membrane, and stored at −20° C. for later use.

### 4) Purification and Preparation of a Clinical Grade Recombinant Adenovirus Gene Therapeutic Agent by Ion-Exchange Chromatography:

AKTA™explorer 100 automatic chromatographic system (Amersham Biosciences (China) Ltd.) was used. The chromatographic conditions included: a 20 ml Q Sepharose XL chromatographic column, 400 ml (20-column volume) elution reagent comprising 1M NaCl, 50 mM Tris/HCl, pH8.0, 5% glycerol, with a linear grads from 0 to 100%. The fraction of recombinant adenoviruses was collected according to the chromatogram. Then, the column was eluted with 0.1M NaOH, and the purified recombinant adenovirus was further desalted, thereby adjusting the pH (pH 8.0). The sample was sterilized by being filtered through a 0.2 μm membrane, packed and stored in a −80° C. freezer in the end.

### Example 3

### Assay for the Titer Value of a Recombinant Adenovirus Comprising Recombinant khp53 Tumor Suppressor Gene, Measurement for the Number of Adenovirons, and Analysis of Gene Expression in Human Malignant Tumor Cells

### 1) Assay for the Titer Value of Recombinant Adenovirus by TCID50 Method:

A suspension of 293IQ cells with a concentration of 1×10⁵ cells/ml was prepared with MEM medium. A 96-well plate was inoculated with the suspension, 100 μl/well. The diluted virus solution was prepared by being diluted in a 10 time series, and utilized to transfect 293IQ cells, respectively. For each row, 100 μl of the diluted virus solution with the same concentration was added into the first 10 wells respectively; and the same volume of MEM containing 2% BCS was added into the 11^{th} and 12^{th} wells at the same row respectively, as negative controls. After cultured at 37° C. in a CO₂ incubator for 10 days, the cells were observed under an inverted microscope and the CPE status was evaluated and recorded. Lastly, the titer value of the recombinant adenovirus was calculated in accordance with the following formula: T=10^{1+d(S-0.5)} IU/ml, wherein d=Log 10(diluted folds)=1, S=the sum of positive ratios from the first dilution (for the value of each well, positive counts for 0.1; negative counts for 0). The difference between the titer values obtained from two parallel experiments should be ≦10^{0.7}=5. The above formula is then shorten as T=10^{S+0.5} IU/ml. As a result, for those two 96-well plates, S values were 8.8 and 8.6, titer values were 2.00×10¹⁰ IU/ml and 1.26×10¹⁰ IU/ml, respectively, and the average titer value was 1.63×10¹⁰ IU/ml.

### 2) Measurement of the Number of Adenovirons by UV Absorption Method:

After addition of an equal volume of 0.2% SDS solution, 250 μl recombinant adenovirus purified in step 4) of Example 2 was cleaved, mixed evenly by shacking, kept in a 56° C. water bath for 10 min, and transiently centrifuged after the temperature dropped to room temperature. The mixture of viral storage solution and 0.2% SDS, with a volume ratio of 1:1 was used as a blank control. The absorbance was measured at 260 nm and 280 nm. wavelength, respectively. The experiments were carried out twice in parallel. The number of virons was calculated in accordance with the following formula: A260×dilution fold×1.1×10¹². The result showed that the viron number of this batch of rAdMH-khp53 was 4.6×10¹¹ VP/ml.

### 3) RT-PCR Detection of p53 Gene Expression:

Human osteosarcoma Saos-2 cells (expressing no p53 gene) and human lung cancer A549 cells (expressing a wild-type p53 gene) (Laboratorial Animal Center of Zhongshan University of Guangzhou) were revived, cultured and plated on 6 cm cell culture plates (two plates for Saos-2 cells; one plate for A549 cells). The Saos-2 cells in one plate were infected with rAdMH-khp53 recombinant adenovirus at 10 MOI. After 10 hours, total RNA was extracted from Saos-2, Saos-2+rAdMH-khp53 and A549 cells by Trizol reagent (invitrogen), according to the method provided in the kit. The first strand of the cDNA of Saos-2, Saos-2+rAdMH-khp53 and A549 cells were synthesized using Fermentas Rebert Aid First Strand cDNA Synthesis Kit (Shenggong Biological Engineering and Technology Service Ltd of Shanghai), according to the method provided in the kit. p53 specific fragment was amplified (the amplification method was same as the method for identifying p53 gene in the part 1) of Example 2) by PCR, wherein the first strand of the cDNA for each type of cells was used as the template. The specific fragment of house-keeping gene GAPDH amplified by PCR was used as inner control, and a blank control was set up as well. The PCR product was observed by agarose gel electrophoresis and photographed under UV, as shown in FIG. 7. Lane 1 presented the RT-PCR result for total RNA of A549 cells, showing the expression of p53 mRNA. Lane 2 presented the RT-PCR result for total RNA of Saos-2 cells, showing no expression of p53 mRNA. Lane 3 presented the RT-PCR result for total RNA of Saos-2 cells infected with rAdMH-khp53 for 10 hours, showing the expression of p53 mRNA, which was resulted by the expression of an exogenous gene. Lane 4 presented a negative control, showing no band, which is due to the absence of template in the RT-PCR system. Lane M presented the DNA molecular weight marker. Lanes 5-7 presented the results of RT-PCR amplification with total RNA of A549, Saos-2, and Saos-2+rAdMH=khp53 cells as the template, respectively, and the specific primers for house-keeping gene GAPDH as the primers, all showing bands. Lane 8 presented the negative control, showing no band, which is due to the absence of template in the RT-PCR system.

### 4) Detection of P53 Protein Expression by SDS-PAGE and Western Blotting:

Total protein was extracted from the cells treated as part 3) using a conventional method, and then quantified. Protein electrophoresis was performed using 15% polyacrylamide gel. A piece of NC membrane and two pieces of Whatman filter paper were cut off and immerged in a transferring buffer for 5 min with two pieces of sponge. After the electrotransferring device was set up, membrane transfer was carried out under 50 mA current overnight. The transferred membrane was washed by PBS for 2 min, followed by being blocked in PBS containing 5% BSA for 30 min. The membrane was incubated for 1 hour on a shaker following the addition of 5 ml of mouse anti-human P53 primary antibody (Shangbo Biological Medical Technology (Beijing) Ltd.) (1:500 diluted by PBS containing 5% BSA). The membrane was washed by PBS for 5 times, 5 min each time. HRP-human P53 secondary antibody (Shangbo Biological Medical Technology (Beijing) Ltd.) was added and diluted 1:1000 (by PBS with 5% BSA). The membrane was incubated in the diluted secondary antibody solution for 30 min on a shaker, and washed by PBS for 5 times, 5 min each time. Then the membrane was placed into DAB color-developing agent, gently shaked at room temperature. The color reaction was observed, and the membrane was washed by water immediately after color of the bands became enough dark (1-3 min). Subsequently, the membrane was transferred into a PBS solution, and photographed after being taken out of the solution. The result was shown in FIG. 8. Lane 1 presented the Western blot result for total RNA of Saos-2 cells, showing Actin protein blot, but no P53 protein blot. Lane 2 presented the result for total protein of Saos-2 cells infected with rAdMH-khp53 for 10 hours, which showed a very big P53 protein blot, indicating large amount of P53 protein expression, and an Actin protein blot with a size similar to that of Lane 1. Lane 3 presented the Western blot result for total protein of A549 cells, showing both a P53 protein blot and an Actin protein blot.

### Example 4

### In vitro and in vivo Growth Inhibition of Human Malignant Tumor Cells by a Recombinant Adenovirus Comprising Recombinant khp53 Tumor Suppressor Gene

### 1) Experiment for Measuring the Survival Rate of Cells by MTT:

A 96-well plate was inoculated with malignant tumor cells (human lung cancer A549 cells, human liver cancer HepG2 cells, human breast cancer MCF-7 cells, human nasopharyngeal cancer HNE-1 cells, human ovarian caner SK-OV-3, human cervical cancer Hela cells, etc) (all available from the Laboratorial Animal Center of Zhongshan University of Guangzhou), respectively. After complete adhesion, the cells were infected with rAdMH (without any exogenous gene) (generated by recombination of the shuttle plasmid without any exogenous gene, i.e. pDC515(io) and the adenoviral genomic backbone, i.e. pBHGfrt(del)E1,3FLP in 293IQ cells, according to the preparation process of rAdMH-khp53 described above), rAdMH-LacZ (with a β-galactosidase gene) (generated by recombination of the shuttle plasmid with a forwardly inserted β-galactosidase gene, i.e. pDC515(io)-LacZ and the adenoviral genomic backbone, i.e. pBHGfrt(del)E1,3FLP in 293IQ cells, according to the preparation process of rAdMH-khp53 described above, wherein the recombinant plasmid pDC515(io)-LacZ was obtained as follows: digesting LacZ gene (SEQ ID NO:12) with the restriction endonulease HindIII and blunting the ends with Klenow enzyme; digesting pDC515(io) with the restriction endonulease EcoRI and blunting the ends with Klenow enzyme; digesting both of above gene fragments with the restriction endonulease BamHI; separating those two DNA fragments by electrophoresis; ligating, transforming and screening the desired plasmid after purification ) and rAdMH-khp53 of different MOI (0, 2.5, 5, 10, 25, 50, 100, 250), respectively. The culture solution was removed 4 days later, and 150 μL (0.5 mg/mL) MTT solution was added into each well. The MTT solution was removed after reacting at 37C for 4 hours, and 150 μL DMSO/well was added. The plate was kept on a horizontal shaker for 10 min. Then A value was measured at the wavelength of 570 nm. The result showed that rAdMH-khp53 possessed a clearly killing function to all malignant tumor cells. For example, in the experiment on human lung caner A549 cells, the number of survived cells in the treated group was significantly decreased, as shown in FIG. 9.

2) Experiment on Treatment of the Transplantation Tumors by rAdMH-khp53 Gene Therapeutic Agent in a Naked Mouse:

Nine naked mice were subcutaneously injected with 0.1 ml PBS suspension containing 5×10⁶ human lung cancer A549 cells at different locations on the anterior part of the back, the right posterior part of the back and the left posterior part of the back, respectively. After tumor inoculation for 6 days, 0.1 ml of 1×10⁹ IU rAdMH-khp53 (the right posterior part of the back), rAdMH-LacZ (the anterior part of the back) and a blank control PBS (left posterior part of the back) were injected into tumor nodules at the different locations (or the locations inoculated with tumors), respectively. The injection was performed every 2 days, and 6 times in total. Volume of tumors was measured before each injection, for a period of 24 days. The result indicated that the volume of the tumors in the gene therapy group was significantly reduced, as shown in FIG. 10. After the experiments, the animals were sacrificed. The organs such as tumors, hearts, livers, spleens, lungs, kidneys and brains, etc were cut off, fixed, embedded in paraffin, and sliced. An immunohistochemical analysis was performed to detect the distribution of the recombinant adenoviruses introduced in target tissues and non-target tissues in animal. The result showed that the structure of tissues such as hearts, livers, spleens, lungs, kidneys and brains, etc maintained well, and there was no clear damage in those tissues. The result also indicated that the recombinant adenoviruses existed in tumor tissues, but not in non-tumor tissues.

1. A recombinant adenovirus shuttle vector, wherein a Kozak sequence and an exogenous gene are inserted between an operator and a PolyA signal sequence in the adenovirus shuttle vector pDC315(io) or pDC316(io), said adenovirus shuttle vector pDC315(io) is shown as FIG. 11(A), and said adenovirus shuttle vector pDC316(io) is shown as FIG. 11(B).

2. A recombinant adenovirus shuttle vector, wherein a Kozak sequence and an exogenous gene are inserted between an operator and a PolyA signal sequence in the adenovirus shuttle vector pDC515(io) or pDC516(io), said adenovirus shuttle vector pDC515(io) is shown as FIG. 11(C), and said adenovirus shuttle vector pDC516(io) is shown as FIG. 11(D).

3. The recombinant adenovirus shuttle vector according to claim 1 or 2, wherein said Kozak sequence is CCACCATGG (SEQ ID NO: 4).

4. The recombinant adenovirus shuttle vector according to claim 1 or 2, wherein said exogenous gene is human p53 gene which comprises the nucleotide sequence set forth in SEQ ID NO:3.

5. An eukaryotic gene expression vector, comprising the sequence of SEQ ID NO:11.

6-10. (canceled)

11. A method for producing the recombinant adenovirus shuttle vector of claim 1 or 2, comprising inserting an exogenous gene linked with a Kozak sequence between an operator and a PolyA signal sequence of an adenovirus shuttle vector in a forward direction, wherein said adenovirus shuttle vector is selected from the group consisting of pDC315(io), pDC316(io), pDC515(io) and pDC516(io).

12-13. (canceled)

14. A recombinant adenovirus obtained by co-transfecting 293IQ cells with the recombinant adenovirus shuttle vector of claim 1 and an adenoviral backbone, said adenovirus backbone is pBHGlox(delta)E1,3Cre shown as FIG. 12(A).

15. A recombinant adenovirus obtained by co-transfecting 293IQ cells with the recombinant adenovirus shuttle vector of claim 1 and an adenoviral backbone, said adenovirus backbone is pBHGfrt(del)E1,3FLP shown as FIG. 12(B).

16. The recombinant adenovirus of claim 14, wherein said Kozak sequence in said recombinant adenovirus shuttle vector is CCACCATGG (SEQ ID NO: 4).

17. The recombinant adenovirus of claim 15, wherein said Kozak sequence in said recombinant adenovirus shuttle vector is CCACCATGG (SEQ ID NO: 4).

18. The recombinant adenovirus of claim 14, wherein said exogenous gene in said recombinant adenovirus shuttle vector is human p53 gene which comprises the nucleotide sequence set forth in SEQ ID NO:3.

19. The recombinant adenovirus of claim 15, wherein said exogenous gene in said recombinant adenovirus shuttle vector is human p53 gene which comprises the nucleotide sequence set forth in SEQ ID NO:3.

20. A recombinant adenovirus, which is the adenovirus with the deposite number of CCTCC V200508.

21. A medicament for treating cancers, comprising the recombinant adenovirus according to any one of claims 14, 15 or 20, and a pharmaceutically acceptable excipient.

22. A method for producing a recombinant adenovirus, comprising co-transfecting 293IQ cells with the recombinant adenovirus shuttle vector according to claim 1 or claim 2 and an adenoviral backbone, wherein if said adenovirus shuttle vector is pDC315(io) or pDC316(io), said adenovirus backbone is pBHGlox(delta)E1,3Cre shown as FIG. 12(A), and if said adenovirus shuttle vector is pDC515(io) or pDC516(io), said adenovirus backbone is pBHGfrt(del)E1,3FLP shown as FIG. 12(B).

23. A method for treating cancers, comprising administering a therapeutic effective amount of the recombinant adenovirus according to any one of claim 14, claim 15 or claim 20, to a patient.

24. The method of claim 23, wherein said cancer is selected from the group consisting of lung cancer, liver cancer, breast cancer, nasopharyngeal cancer, ovarian cancer and cervical cancer.
